(19) United States
(12) Patent Application Publication
(10) Pub. No.: US 2008/0254137 A1
(43) Pub. Date: Oct. 16, 2008

(54) Treatment of autoimmune disorders using detoxified cobratoxin

(76) Inventors: Laurence N. Raymond, Plantation, FL (US); Paul F. Reid, Plantation, FL (US)

Correspondence Address: Robert J. Van Der Wall;Gables One Tower, Suite 1275, 1320 South Dixie Highway, Coral Gables, FL, 33146, US

(21) Appl. No.: 11/784,607

(22) Filed: Apr. 10, 2007

(51) Int. Cl. A61K 35/58; A61P 1/00; A61P 19/02; A61P 21/00; A61P 37/00

(52) U.S. Cl. 424/542

(57) ABSTRACT

Autoimmune disorders are widespread diseases with many manifestations. Immune regulatory dysfunction is central to the progress of these diseases. The control this dysfunction can be achieved through intervention in the immune pathways. The balance of regulatory cytokines is central to a correctly functioning immune system and is a target for therapeutic intervention. Herein is described the induction of the regulatory cytokines, interferon gamma and interleuking 27, as a method to treat autoimmune diseases and a method by which such regulatory cytokines can be induced using a detoxified cobra neurotoxin composition.

### BACKGROUND OF THE INVENTION

1. Field of the Invention

The present invention relates generally to the field of therapy for the autoimmune disease Multiple Sclerosis (MS), as well as to other autoimmune diseases which have an inflammatory basis. In autoimmune pathology there are three pathways that can become skewed to produce a “self” destructive disease. A change in the pathology inherent in autoimmune diseases can be modified by the induction of expression of a down-regulating component in one of those pathways. The present invention relates specifically to the induction of interferon gamma (IFNγ) and IL-27 production in T cells from subjects with autoimmunity and other cells capable of such production by the administration of a composition of a detoxified neurotoxin derived from the venom of the cobra snake of the species Naja.

2. Description of the Prior Art

There are three independent pathways which affect inflammation: IL-12/IFN gamma; IL-4/IL-5/IL-13 and IL-23/IL-17 (Iwakura Y and Ishigame H; 2006; Journal of clinical investigation 116:1218-1222). The introduction of foreign material (an antigen) and interaction of that antigen with a naive CD4+ T cell results in that T cell maturing along one of those three pathways. Which pathway is activated is dependent upon the type of antigen. If the antigen is one that is handled by a cell mediated immune response, the cell matures under the influence of IL-12 produced by macrophages, into the T helper subset, Th1 bearing the IL-12 receptor (IL-12R). The Th17 subset is negatively regulated by the Th1 subset by IFN-γ, and by the Th2 subset through IL4 and by T-reg cells through IL10 and TGF-β1. In spite of this regulation, the Th17 subset has been implicated in the development and exacerbation of autoimmune disease, especially EAE, the animal model for multiple sclerosis, and other inflammatory diseases as inflammatory bowel syndrome. (Yen D et al; 2006, J. Clin. Invest. 116: 1310-1316; Harrington L E et al, 2005, Nat Immunol 6:1123-1132).

The function of the Th17 cell subset, through the secretion of IL-17, is considered to be the control of inflammation (Harrington LE et al., 2005 Nat Immunol 6:1133-1141; Park H et al., 2005, Nat Immunology 6:1133-1141). IL-17 is a family of six molecules, all produced by the Th17 subset. IL-17, also known as IL-17A, and IL-17F are best known though whether all these molecules have the same or disparate activity is not currently known. The presence of IL-17 is a consistent finding in autoimmune disease and is associated with pathology in chronic inflammation and autoimmune disease in general: rheumatoid arthritis (Chabaud M et al, 2000, Cytokine 12:1092-1099); inflammatory bowel disease (Ulcerative colitis and Crohn's Disease) (Fujino S, 2003, Gut 52:65-70); autoimmune arthritis (Ishigame H et al, 2006, Ernst Schering Res Found Workshop 56:129-153): systemic autoimmune disease (Lohr J et al, 2006, J Exp Med 203: 2785-2791).

There are several lines of evidence which indicate that IL-17 is implicated in autoimmune diseases and that IFN-γ is implicated as a regulator of IL-17. Until recently IFN-γ was considered to have a supporting/causative role in autoimmune disease, especially Experimental Autoimmune Encephalomyelitis/Multiple Sclerosis (EAE/MS). Administration of exogenous IFN-γ resulted in increases in the severity of MS (Panitch H S, 1987, Neurology 37:1097-1102) and EAE (Sun D et al, 2004, Neuropathol Appl Neurobiol, 30:374-384). On the other hand, mice deficient in IFN-γ or IFN-γR were found to exhibit a chronic EAE course similar to wild type controls (Willenborg D O et al, 1996, J Immunol 157:3223-7; Ferber I A et al, 1996, J Immunol 156:5-7), suggesting a lack of significant impact on disease development and maintenance by IFN-γ.

Using IFN-γ receptor deficient mice (IFNγR^{-/-}) it has been determined that IFNγ is not required for EAE expression, but is essential for down-regulation of EAE (Willenborg et al, 1996, J Immunology 157:3223-3228; Willenborget al, 1999 J Immunology 163:5278-5286). In IL-17 deficient mice, EAE development was significantly suppressed, with delayed onset, reduced severity, ameliorated histological scores and early recovery. In addition, adoptive transfer of cells to wild type mice inefficiently induced EAE in recipient mice (Komiyama et al, 2006, 177:566-573). Mice lacking the ligand binding chain of the IFN-γ receptor develop severe and fatal human myelin oligodendrocyte glycoprotein (MOG) peptide-induced EAE, while controls are resistant to induction of disease (Willenborg et al, 1996, J Immunology 157:3223-3227). Passive transfer of the disease with MOG-specific T-cells to IFN-γ mice results in severe and terminal EAE. The same cells when transferred to IFN-γ normal mice results in severe disease from which all recipients recover. IL-17 mRNA expressing mononuclear cells are higher in the blood of patients with MS during exacerbation than during remission; the levels in CNS were higher than those found in blood. (Matusevicius D et al, 1999, Mult Scler 5:101-104). Administration of anti-IL-17 antibodies after induction of EAE in mice, but prior to EAE onset results in delay of onset in comparison to controls. Administration of anti IL-17 antibodies after the development of EAE symptoms resulted in the reversal of progression of disease (Park H et al, 2005 Nat Immunol 6:1133-1141).

IL-27, produced by antigen presenting cells (APCs) has, along with the Th1 and Th2 subsets, also has important immuno-regulatory activities which exert an antagonistic influence over IL-17. The IL-27R is expressed on T and B cells as well as myeloid cells. Mice lacking the IL-27 receptor develop severe CD4+ dependent, neuroinflammation, which is accompanied by a substantial IL-17 response, when chronically infected with Toxoplasma gondii (Stumhofer J S et al, 2006, Nat Immunology 9:937-945). Also, mice lacking the IL-27R have increased susceptibility to EAE, and produce high levels of IL-17 producing cells (Batten M et al, 2006, Nature Immunology 7:929-936). IL-27 suppresses IL-6/TGF-β mediated T cell proliferation of Th17 subset cells (Batten M et al, 2006, Nature Immunology 7:929-936; Stumhofer J S et al, 2006, Nat Immunology 7:937-945) and inhibits the production or IL-17 by CD4+ T cells (Yoshimura T et al, 2006, J Immunology 177:5377-5385). As a consequence of it's influence on the Th17 subset and IL-17 production, IL-27 provides a potential control mechanism for IL-17 in cases of autoimmune inflammation. Additionally, IL-27 has a positive effect on the differentiation of naive T cells into the Th-1 subset, a producer of IFN-γ. When IL-27 control is absent, as demonstrated in mice lacking the IL-27 receptor (I127ra−/−mice) which have EAE, there is an exacerbated level of disease in comparison to wild type mice (Batten et al., Nature Immunology., 2006, 7:929-936).

Native cobra venom (CV) and neurologically active cobratoxin (CT) isolated from cobra venom can be detoxified by incubation with hydrogen peroxide. Detoxification is accomplished by a modified Fenton reaction as taught by Sanders U.S. Pat. No. 3,888,977, that allows the safe administration of neurotoxin proteins.

### SUMMARY OF THE INVENTION

Bearing in mind the foregoing, it is the principal object of the present invention to provide a method for the treatment of Multiple Sclerosis and other autoimmune diseases by the parenteral administration of modified cobratoxin or modified cobra venoms containing cobratoxin. Such administration resulting in the induction of IFN-γ and IL-27 expression which alone and in concert has/have suppressive effects on the production of Th17 subset cells and the production of IL-17, such activities having an ameliorating effect on autoimmune disease.

Other objects and advantages will become apparent to those skilled in the art upon reference to the following description and the appended claims.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

An autoimmune disease results from the immune system's recognition of host structures as foreign and its mounting a response to them. What triggers these events is not entirely clear and external factors or events are suspected to be involved. Many infectious agents have epitopes that mimic host structures and autoimmune reactions often occur following a viral or bacterial illness. The cause of some autoimmune diseases are not clearly understood as exemplified by Multiple Sclerosis, an autoimmune disease of the central nervous system, and adult onset diabetes.

The formation of E-rosettes, a function of T cells from peripheral blood, and a method used for T cell enumeration, is decreased by 30%-40% by carbamylcholine chloride, a cholinergic antagonist, indicating the expression of nAchR on at least a subset of human T cells (Mizuno et al., 1982). Consequently T-cell functions can be influenced by anticholinergics including peptide neurotoxins, an important aspect in autoimmune diseases. Including the inhibition of viral replication in immune cells, and viral inhibition even in the absence of acetylcholine receptors, and reported use of modified venoms in patients with rheumatoid arthritis (Montsedeoca et al, 1987) may suggest a general use in immune system disorders. On an equivalent weight basis, modified cobratoxin (mCT) was found to prevent the development of acute and chronic EAE in 11 of 12 rats while modified whole venom was found to prevent the development of acute and chronic EAE in 6 of 12 rats (Mohamed et al., Biomed Sci Instrum, 2006, 42:399-404). The mechanism for this activity is considered to be the action of components of mCV, of which mCT comprises about 15%, or the action of mCT alone, on CD4+ T cells resulting in the production of IFN-γ, depressing the development of EAE.

Detoxified (modified) cobra venom (mCV) and detoxified cobratoxin (mCT) has been demonstrated to cause the production of IFN-γ in vitro by peripheral blood mononuclear cells and BHK-21 cells and in vivo by parenteral administration (Sanders, unpublished results). Based upon gene micro-array data, exposure of PBMC from normal donors to modified cobratoxin (mCT) resulted in a 23 fold increase in IFN-γ as well as a 21.2 fold increase in IFNγ inducible protein. These increases are referenced to the mRNA levels determined in non-stimulated cells. Testing of modified cobra venom (mCV) in on PBMC from “normals” indicated a 6.1 fold increase in IFNγ and 6.1 fold increases in IFNγ inducible protein and provides a mechanism by which mCT and mCV exert their antiviral effects.

When PBMC from MS patients were exposed to mCT, there was an increase in the expression of mRNA for IFNγ by a factor of 2.3, and IL27 by a factor of 3.4. Interestingly, IL-27 was increased in brain tissue from MS lesions obtained at autopsy by 4.5 fold when exposed to mCT, a location where the suppression of the inflammatory reaction is most relevant.

Thus the use of mCT to induce the production of IL-27 and IFNγ would result in activity which would have a direct suppressive effect on the Th17 subset of T-cells that produce IL-17. An increase in IFNγ by the Th1 subset would also exert a negative effect on Th17 subset development while an increase in IL-27, indicating an action on APC, would also have a negative effect on the Th17 subset. This would offer a modicum of control over a pathologic system. More succinctly, there are hypothesized to be two active and opposing immunological controlling pathways in MS/EAE autoimmune disease: 1.) A pathologic pathway due to a lack of strict control over propagation of the Th17 CD4+ T cell subset, resulting in an over production of IL-17 and in the development and support of autoimmune diseases such as MS/EAE, and, 2.) A palliative pathway which exerts partial control on IL-17 production by secretion of IFN-γ by the Th1 CD4+ T cell subset, and /or by other T cells and other IFNγ secretion competent cells. This pathway can be activated by the action of mCT. The increase in IFN-γ and IL-27 production exerts a negative effect on the differentiation of the Th17 CD4+ T cell subset and depression of IL-17.

### EXAMPLE 1

### Inhibition of Acute Onset EAE

For the study female Lewis rats were divided into groups of 12 rats each. Two groups received subcutaneous injection of 200 mg guinea pig myelin basic protein (MBP) plus complete Freund's adjuvant (CFA) and treated with either modified cobratoxin or modified cobra venom. The control group received only subcutaneous injection of 200 mg guinea pig myelin basic protein (MBP) plus complete Freund's adjuvant (CFA). An acute phase study was run for 28 days post EAE induction.

All treated animals were receiving the treatment as three doses per week for three weeks prior to EAE induction. Each single dose of modified cobratoxin or cobra venom was 0.2 mg and was given subcutaneously.

All animals were examined for behavioral deficits daily. The examinations were by two individuals who were blinded as to the injections they received.
Pender Scores as follow:
Score 0 No Symptoms
Score 1 Tail Weakness
Score 2 Tail Paralysis
Score 3 Hind limb weakness
Score 4 Forelimb Weakness
Score 5 Hind limb Paralysis
Score 6 Forelimb Paralysis

For tissue examination, each animal was sacrificed under perfusion with saline and Halothane anesthesia. Spinal cord and brain tissues fixed in formalin and embedded in paraffin, sectioned in 2-4 micrometer in thickness. The section stained with Hematoxylene and Eosin and examined using light microscope for the presence of perivascular lymphocyte infiltrate inflammation and graded as no inflammation, mild, moderate and severe as described by (Mohamed, 2004).

At the 28^{th} day all had been sacrificed and perfusion fixed, histological examination shows no signs of perivascular cuffing in all of modified cobratoxin group except one animal, who clinically was sick, showed mild perivascular cuffing in the spinal section. The modified cobra venom group showed no signs of perivascular cuffing in six animals and those animals that were clinically sick showed mild to moderate perivascular cuffing in the spinal and brain sections. All control animals were symptomatic at 11-24 days.

### EXAMPLE 2

### Inhibition of Chronic EAE

For the study female Lewis rats were divided into groups of 12 rats each. Two groups received subcutaneous injection of 200 mg guinea pig myelin basic protein (MBP) plus complete Freund's adjuvant (CFA) and treated with either modified cobratoxin or modified cobra venom. The control group received subcutaneous injection of 200 mg guinea pig myelin basic protein (MBP) plus complete Freund's adjuvant (CFA) used as a control animal models of EAE for the acute and relapsing stages respectively. A chronic phase study was run for 70 days post EAE induction.

Animals were maintained on 0.2 ml of a drug once a week for the next five weeks. Animals were examined for behavioral deficits and weighed twice a day by two individuals. One animal showed symptoms day 7 to 20 when treated with modified cobratoxin whereas six animals showed symptoms day 11 to 27, when treated with modified cobra venom. All control animals were symptomatic at 11-24 days.

Histological examination at day 70 revealed six animals with no inflammation and six animals showed different degrees of perivascular inflammation from the modified venom group. In the modified cobratoxin group histological examination revealed only one animal with severe perivascular inflammation.

### EXAMPLE 3

### Induction of IFN-Gamma Production in PMBCs of MS Patients

Methods: T-cells were collected from waste whole blood collected under IRB-approved protocol. In brief, blood was placed in Becton Dickenson Cell-Prep Tubes (CPT) and centrifuged to separate PBMC's from RBC's. PBMC's were collected, washed 3× with warm PBS and cultured on plastic overnight at 37° C./5% CO₂ in RPMI-1640 containing protein and cytokine supplementation to remove macrophages. Remaining PBMC's were again washed 3× with PBS and mixed with DynaBeads. The T-cells were then isolated by magnetic bead separation. The purified T-cells were washed 3× with warm PBS and cultured for 2 days as described above to acclimate the T-cells to culture conditions. T-cell purity was confirmed by flow cytometry.

Assay: 5.0E5 T-cells from each sample were removed from the culture and divided into 2 parallel cultures, one containing 2.5E5 cells with 10 ug/ml of modified venom in 2.5 ml serum/cytokine-free RPMI-1640 and the other containing 2.5E5 cells with 2.5 ml serum/cytokine free medium. Cells were then placed in culture for 18 hours. Using a CASY1TTC Cell Analyzer to determine remaining cell concentration, 1.25E5 cells were harvested, washed 3× with warm PBS-T and centrifuged into a pellet. After removal of the final wash supernatant, the pellet was resuspended in RNA-preservation solution and snap-frozen in liquid nitrogen and stored at −130° C. until Micro-array analysis.

Micro-array Analysis: Samples were transported on dry ice to the microarray facility and processed per standard protocol as established and published by Affymetrix. Briefly, RNA is harvested, placed on HG-U133Plus2.0 array chips. After hybridization, chips were placed into Affymetrix chip reader and analyzed.

Statistics: The control samples cultured in parallel without drug exposure were used to subtract background from the test samples. Once background subtracted, the individual data were combined and subjected to statistical analysis. The data was reported as the means of the samples that met 90% confidence (p=0.10). IF-gamma was increase by a factor of 2.3 fold over controls.

### EXAMPLE 4

### Induction of Il-27 Production in PMBCs of MS Patients

Method of analysis was as described for Example 3 and the responses in Il-27 expression was determined to increased by a factor of 3.4 over controls.

### EXAMPLE 5

### Induction of IL-27 Production in Brain Histocultures from MS Subjects

MS lesion and non-diseased white matter were obtained from 5 individuals (5 female, 57 year median age) through rapid post-mortem collection or MRI-guided biopsy (excess diagnostic tissue). Tissue was processed into 3-5 mm³ fragments and placed on a collagen sponge soaked with and floating in culture medium with or without mCT, at the liquid-air interface. The medium was changed daily and after 72 hours continuous exposure to mCT, RNA was collected for microarray analysis. The responses in I1-27 expression was determined to increased by a factor of 4.5 over controls.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

What is claimed is:

1. A method of treatment of animals suffering from autoimmune disorders through the induction and expression of regulatory cytokines.

2. A method of claim 1 where the induction and expression of interferon gamma is upregulated.

3. A method of claim 1 where the induction and expression of interleukin-27 is upregulated.

4. The method of claim 1 wherein the regulatory cytokines are upregulated through the use of a composition comprising detoxified cobra venom containing cobratoxin.

5. The method of claim 4 wherein the composition is detoxified cobratoxin.

6. The method of claim 1 wherein the autoimmune disorder is Multiple Sclerosis.

7. The method of claim 1 wherein the autoimmune disorder is Rheumatoid Arthritis.

8. The method of claim 1 wherein the autoimmune disorder is systemic autoimmune disease,

9. The method of claim 1 wherein the autoimmune disorder is inflammatory bowel syndrome.

10. The method of claim 1 wherein the autoimmune disorder is autoimmune induced Diabetes.

11. A method of treatment of animals suffering from autoimmune disorders through the induction and expression of one of interferon gamma and interleukin-27 is upregulated using a composition comprising detoxified cobra venom containing cobratoxin.

12. The method of claim 11 wherein the autoimmune disorder is Multiple Sclerosis.

13. The method of claim 11 wherein the autoimmune disorder is Rheumatoid Arthritis.

14. The method of claim 11 wherein the autoimmune disorder is systemic autoimmune disease,

15. The method of claim 11 wherein the autoimmune disorder is inflammatory bowel syndrome.

16. The method of claim 11 wherein the autoimmune disorder is autoimmune induced Diabetes.
